(19) United States
(12) Patent Application Publication
(10) Pub. No.: US 2017/0190636 A1
(43) Pub. Date: Jul. 6, 2017

(54) METHOD FOR PRODUCING PRODUCT OLEFINS BY CATALYTIC DEHYDRATION OF SUITABLE REACTANTS

(71) Applicant: Linde Aktiengesellschaft, München (DE)

(72) Inventors: Petra Rehak, Dresden (DE); Florian Winkler, München (DE)

(73) Assignee: Linde Aktiengesellschaft, Munchen (DE)

(21) Appl. No.: 15/314,183

(22) PCT Filed: May 28, 2015
(86) PCT No.: PCT/EP2015/061860

§ 371 Date: Nov. 28, 2016

(30) Foreign Application Priority Data
May 28, 2014 (EP) 14001870.6

(51) Int. Cl. C07C 1/24; C07C 1/20

(52) U.S. Cl. CPC C07C 1/24; C07C 1/20

(57) ABSTRACT

The invention relates to a method for producing product olefins by catalytic dehydration of suitable reactants, comprising the steps of feeding a educt stream comprising an alcohol-water mixture into a dehydration unit, wherein the alcohol-water mixture comprises at least one alcohol and water, and converting the reactants contained in the educt stream in the dehydration unit by catalytic dehydration to a mixed reaction product stream, the dehydrating conditions in the dehydration unit being selected such that the dehydration of the alcohol comprises only a low conversion of at least one alcohol used to the desired at least one product olefin, such that the unreacted at least one alcohol in the mixed reaction product stream comprises an alcohol content ranging from 20 wt %-80 wt %.

The invention relates to a method for producing product olefins by catalytic dehydration of suitable reactants, in particular by catalytic dehydration of alcohols and alcohol mixtures.

The dehydration of alcohols to form product olefins by elimination of water in the presence of a catalyst is a known reaction. For example, by dehydration of butanol, 1-butene (in the literature also referred to as 1-butylene) as the main product and 2-butene (or 2-butylene) as a by-product can be produced. The dehydration is a highly endothermic reaction.

US 2011/0213104 A1 describes a method for producing ethylene-butylene copolymers from renewable resources. The ethylene is produced by means of a dehydration reaction of ethanol, which is provided by a fermentation of sugar. Furthermore, butylene is produced by a dehydration reaction, wherein the starting material butanol is provided by a fermentation of sugar or by a chemical reaction of the above-mentioned ethanol. Alternatively, butylene can be produced by a dimerization reaction of the ethylene provided by the dehydration reaction described above. The reaction conditions of the dehydration reaction of the butanol are selected such that a selectivity of 77.5% of 1-butylene and 20% of 2-butylene is achieved in the final product (based on the molar amount of the butanol). Furthermore, a separation of the dehydration products obtained from the butanol by means of a distillation is described, wherein unreacted butanol can be separated and fed into the dehydration reactor.

From EP 2 374 781 A1 a method is known in which isobutanol is simultaneously dehydrogenated and submitted to a skeletal isomerisation, wherein essentially the olefins with the same number of carbon atoms are formed. These essentially represent a mixture of n-butenes and isobutene. The method comprises feeding a stream containing isobutanol and optionally water and an inert component into a reactor and contacting it therein with a catalyst under suitable conditions. After an optional removal of water and the inert components, the effluent stream of the reactor can be separated into a stream containing n. butenes and a stream containing isobutene. The latter can be recycled into the reactor. A comparable method is known from WO 2011/113834 A1.

WO 2013/032543 A1 discloses a method and an apparatus for dehydrogenating biogenous 1-alcohols to 1-alkenes with high selectivity. The 1-alkenes can advantageously used for producing diesel and kerosene with a high inflammation point. The 1-alkenes can also be converted to thermally stable lubricants.

The invention has the object of providing an economical process for the production of product olefins by catalytic dehydration of suitable reactants. Particularly, the invention should disclose an energetically advantageous method with high selectivity.

This object is solved by a method having the features of independent claim 1.

The process for the preparation of product olefins by catalytic dehydration of suitable reactants according to the invention comprises the steps of feeding an educt stream which essentially comprises an alcohol-water mixture, the alcohol-water mixture comprising at least one alcohol and water, into a dehydration unit, and converting the reactants contained in the educt stream in the dehydration unit by catalytic dehydration to form a mixed reaction product stream, wherein the dehydrating conditions in the dehydration unit are selected in such a way that the unreacted at least one alcohol in the mixed reaction product stream comprises an alcohol content ranging from 20 wt %-80 wt %, in particular in the range of 20 wt %-60 wt %, particularly preferably in the range of 20 wt %-40 wt %. The wt % (percent by weight) indication refers to the proportion of alcohol in the mixed reaction product stream in relation to the other compounds present in the mixed reaction product stream.

The inventive method thus allows, via the selection of suitable reaction conditions, a low conversion of the alcohols used to the desired at least one product olefin. By dehydration with a low conversion, a high selectivity is achieved with respect to the formation of the desired product olefin. The isomers of the desired product olefin formed as by-products are produced to a very small extent at low conversions.

The mixed reaction product stream according to the invention comprises the desired at least one product olefin (olefins are also known as alkenes), optionally at least one isomer of the desired product olefin, unreacted alcohol, dialkyl ethers formed during the dehydration, water, and other by-products formed during the dehydration, such as for example carbon monoxide, carbon dioxide, hydrogen, methane and other alkanes and olefins.

The educt stream according to the present invention essentially comprises as reactants alcohols or alcohol mixtures, in particular higher alcohols and alcohol mixtures. Furthermore, the educt stream may comprise dialkyl ethers, formed in the dehydration and being recycled, as reactants. Dialkyl ethers include symmetrical dialkyl ethers (which are formed from a reaction of two identical alcohols) and unsymmetrical dialkyl ethers (which are formed from a reaction of two different alcohols). Examples of symmetrical dialkyl ethers are diethyl ether, dipropyl ether, dibutyl ether, dipentyl ether or dihexyl ether. Examples of unsymmetrical dialkyl ethers are butyl hexyl ether or butyl pentyl ether.

The educt stream may comprise synthetic alcohols which either contain water or to which water has been added. Alternatively, the educt stream can also be provided by fermentation. By a fermentation of biomass, such as for example sugar, various alcohols can be obtained as alcohol-water mixtures. Preferably, the alcohol-water mixture forming during fermentation can be enriched to an alcohol-water mixture with a higher alcohol content in an enrichment unit in which water is separated.

In one embodiment, the educt stream comprises an alcohol content of 5 wt %-98%, in particular from 40 wt %-96 wt %, particularly preferably from 75 wt %-95 wt %.

In one embodiment of the invention, alcohols with a carbon content of 3 to 8 carbon atoms (that is, alcohols from the group of C₃-C₈ alcohols), in particular 4 to 6 carbon atoms (that is, alcohols from the group of C₄-C₆ alcohols) are used. The alcohols used comprise both linear and branched alcohols. The alcohols used include alcohols with one or more (e.g. diols such as butane diol, pentane diol or hexane diol) OH functional groups. In particular, without limiting the inventive method thereto, n-butanol, iso-butanol, tert-butanol, 1,4-butane diol, 1,3-butane diol, 2,3-butane diol, n-pentanol, n-hexanol as well as their structural isomers (such as e.g. 2-pentanol, 3-pentanol, 2-methyl butane-2-ol, 3-methyl butane-1-ol, 3-methyl butane-2-ol, 2,2-dimethyl propane-1-ol, 2-hexanol, 3-hexanol, 2-methyl pentane-1-ol, 3-methyl pentane-1-ol, 3-methyl pentane-2-ol, etc.) are used singly or in a mixture. The use of C₄ alcohols, in particular n-butanol, is particularly preferred.

The dehydration unit is adapted to provide alkenes from the reactants of the educt stream fed in. The dehydration unit can comprise one or more reactors that are sequentially connected for carrying out the dehydration. The Dehydration can be performed isothermally as well as adiabatically.

In one embodiment of the invention, the mixed reaction product stream is cooled by a cooling unit subsequent to the dehydration, to form mixed reaction product stream with several phases, so that the mixed reaction product stream comprises an aqueous phase and an organic liquid phase, and wherein subsequently the mixed reaction product stream is fed into a phase separation unit in which a phase separation of the aqueous from the at least one organic liquid phase is carried out. Subsequently, the aqueous phase and the at least one organic liquid phase are separated from the mixed reaction product stream with several phases. The separation of the phases, for example, can be done in the simplest way by means of centrifugal force, e.g. in a separator, or by means of gravity, e.g. in a mixer-settler apparatus.

The term “aqueous phase”, in the context of the invention, denotes the phase that comprises the major proportion of the water produced in the dehydrogenation reaction, wherein, depending on the nature of the educts used, the aqueous phase also comprises unreacted alcohols and dialkyl ethers formed during the reaction.

The term “organic liquid phase”, in the context of the invention, denotes a liquid phase that contains, depending of the nature of the educts used and the conditions of phase separation, unreacted alcohols, dialkyl ethers formed in the reaction, product olefins, their isomers, and by-products. Also, the organic-liquid phase can contain a small amount of the water formed in the dehydration reaction.

After the phase separation, the organic-liquid phase is passed into at least one separation unit, wherein unreacted alcohols and dialkyl ethers formed in the dehydration are separated such that they form a recyclable separation stream that is fed to the educt stream.

Furthermore, in addition to the recyclable separation stream, also the at least one desired product olefin and optionally isomers of the produced product olefins are separated from the other by-products. The product olefins mentioned thus form an olefin stream which is withdrawn from the separation unit. The by-products are separated in the separation unit from the organic phase and are withdrawn. Resulting isomers of the product olefin(s) can also be recycled to the educt stream or withdrawn from the plant.

By recycling the recyclable separation stream which does contain alcohols and diethyl ethers, it is possible that unreacted alcohol and dialkyl ethers formed in the dehydration, which are contained in the organic liquid phase after phase separation, are not removed from the reaction cycle but are instead available to a further reaction in the dehydration unit. Thus, the overall reaction of the alcohol to the desired product olefin may be increased. The dialkyl ethers contained in the organic liquid phase are intermediate products which are formed in a dehydration and which can be converted by a further dehydration to give the desired product olefin. These dialkyl ethers are therefore reactants as well which are suitable to be converted by means of a dehydration step to give the desired product olefins. By feeding the dialkyl ethers into the educt stream, the overall conversion of the desired product olefins is also increased. The intermediate products formed in a dehydration reaction (that is, the dialkyl ethers) are recycled.

The educt stream may exclusively comprise an alcohol-water mixture when starting the plant, wherein further reactants, such as dialkyl ethers, are fed only after a first pass, as described above, into the educt stream.

Subsequently to the separation of the aqueous phase from the organic liquid phase, in one embodiment the aqueous phase is fed into the educt stream via a concentration in which the at least one alcohol and dialkyl ether formed in the dehydration are enriched.

In the method known from the prior art, generally the reaction water which is present after the dehydration of the alcohol water mixture in the mixed product stream is separated. Thus, a portion of the unreacted alcohol, which is dissolved in the reaction water, is lost. Via recycling of the unreacted alcohol present in the aqueous phase and the dialkyl ether contained in the aqueous phase, these reactants are available for a further dehydration and can thus be converted to the desired alkene. The overall conversion of the used alcohol is thus increased with respect to the resulting and desired product olefin by this recycle to the reaction cycle. Furthermore, no waste water which must be cleaned with high effort or disposed of is formed in the dehydration. Also, the separation of the aqueous phase from the organic liquid phase (and thus the separation of the reactants dissolved therein) is done in a simple manner, such as by means of a decanter, that is without causing a large energy or equipment expense.

The enrichment or concentration of the aqueous phase can be done for example by distillation, pervaporation or extraction in an enrichment unit. Such an enrichment unit can, for example, also be an enrichment unit in which alcohol from a fermentation is enriched or concentrated. Such enrichment units are normally present in methods which provide the starting material (i.e. the alcohol to be converted) from a fermentation step. Thus, it is possible to combine the aqueous phase with the alcohol-water mixture from a fermentation and to supply them to a single enrichment unit. The alcohol-water mixture enriched there can then subsequently be combined with the educt stream or then forms the educt stream and is fed into the dehydration unit. Therefore, the equipment and energy expense are moderate.

In particular, this applies when a fermentation process is used in which a fermenter broth is present, since the aqueous phase can then be added to the fermenter broth. The alcohol concentration in the aqueous phase is in the order of magnitude of the alcohol concentration in the fermenter broth and the mass flow of the aqueous phase is relatively low compared to the fermenter stream. The mass flow of the aqueous phase is dependent on the operation of the plant, sometimes reaching only about 1/12 compared to the fermenter stream. Also with a continuous fermentation process, for example, apparatus is provided which is intended for concentration. For example, the aqueous phase can be added into the stripper or directly into the fermenter broth. The means for enrichment in the various fermentation methods are known in the art.

Alternatively, the alcohol to be converted or the alcohol mixture to be converted can also be produced synthetically, such as from synthesis gas by means of suitable catalysts. This results usually in alcohol mixtures that are fed to the educt stream either directly or after processing (e.g. distillation). Again, the aqueous phase can be fed in at a suitable location, for example into the distillation.

The phase separation of the aqueous phase from the organic liquid phase thus allows a recycle of unreacted alcohols and/or dialkyl ethers formed in the dehydrogenation reaction in a simple manner. The recycling of the alcohol and the dialkyl ethers allows for a better utilization of the alcohol used as the starting material for the production of a product olefin. The alcohol used and the resulting dialkyl ether can be recycled via the recycling of the aqueous phase described above and/or the recycling of the separation stream into the dehydration unit. By recycling the dialkyl ethers formed as intermediate products and the unreacted alcohol, particularly low and easier achievable conversions can be run in the dehydration unit without getting any economic disadvantages.

Thus, by recycling the intermediates (dialkyl ethers) and of the unreacted alcohol and by the presence of a low conversion in the dehydration unit both the selectivity and the yield of the desired product olefin are increased.

In embodiments of the invention, at least partial streams of the aqueous and/or the organic liquid phase can be recycled into the educt stream. Preferably, the aqueous phase after an enrichment, and/or a separation stream that has been recovered from the organic liquid phase and that at least partially contains the alcohols and/or the dialkyl ethers from the mixed reaction product stream is recycled to the dehydration unit. Thus, the alcohols and/or the dialkyl ethers from the mixed reaction product stream may be fed into the dehydration again. Which of the possible recycles are realized depends on the composition of the mixed reaction product stream and follows economic considerations.

In addition to the organic liquid phase and the aqueous phase in the dehydrogenation additionally also an organic gaseous phase may be formed. Whether an organic gaseous phase is formed depends on the alcohols used as starting materials, the extent of the reaction of the alcohols and the reaction conditions of dehydration and the conditions in the phase separation unit (in particular pressure and temperature). The proportion of the compounds contained in the aqueous or in the at least one organic phase (such as e.g. unreacted alcohol or dialkyl ether) is thus dependent on the reaction conditions and the nature of the alcohols used. With respect to an explanation of the phase separation conditions, reference is made to passages below. The type of separation and the further use of the separated phases are also determined thereby. This is one of the standard tasks of an expert in the field of olefin production and olefin separation and can readily be performed by him.

For example, if higher alcohols (e.g. butanols, pentanols or hexanols) or mixtures thereof are used as educt, it is possible that the mixed reaction product stream comprises in addition to the aqueous phase an organic liquid phase. The organic liquid phase comprises unreacted alcohol, dialkyl ethers resulting from the dehydration, at least one product olefin and optionally the corresponding isomers thereof. The aqueous phase substantially comprises unreacted alcohol.

In this embodiment also a gaseous phase is present which, however, includes only by-products. The organic gaseous phase may, apart from impurities, consist entirely of by-products such as carbon monoxide, hydrogen, carbon dioxide, methane and alkanes. The organic gaseous phase can, however, in addition to the by-products also contain the product olefin(s) and isomers of the product olefin(s). It is also possible that, furthermore, unreacted alcohols and dialkyl ethers are contained in the organic gaseous phase. By-products can be separated in the phase separation and/or in the separation unit and/or in an isomer separation unit and withdrawn. Alternatively or additionally, by-products such as CO₂ can be separated using a CO₂ wash prior to the introduction into the separation unit.

After phase separation of the aqueous, the organic liquid and the organic gaseous phase, the organic gaseous phase is then passed to a separation unit for separation of individual components. This can for example be done by means of a compressor. In the separation unit, the product olefin(s) (and, optionally, isomers thereof formed) is/are separated off, wherein an olefin stream is formed which is withdrawn from the separation unit. The aqueous phase and the organic liquid phase are treated as described above.

For example, if an alcohol mixture of lower alcohols (e.g. n- or iso-propanol) and higher alcohols (e.g. butanols such as n-, iso- and/or tert-butanol and/or e.g. pentanols such as 1-pentanol and isomers thereof) is used as the educt, the mixed reaction product stream comprises—in addition to the aqueous phase—an organic liquid phase and an organic gaseous phase. The aqueous phase case comprises unreacted alcohols (e.g. butanols or propanols) and a proportion of dialkyl ether. The organic liquid phase comprises a proportion of unreacted alcohols, a proportion of dialkyl ether and at least one product olefin, such as 1-butene, and optionally corresponding isomers of the at least one product olefin. The organic gaseous phase comprises at least one product olefin (e.g. propene) as well as educts (alcohols) and dialkyl ether.

Particularly when using higher alcohols, by cooling the mixed reaction product stream and the associated formation of a two-phase liquid mixed reaction product stream, advantageously the two-phase mixed reaction product stream can be separated in a simple manner by means of a phase separation unit into said aqueous phase and said organic liquid phase. The separation of the aqueous phase from the organic liquid phase can be done, for example, by means of a decanter.

The method according to the invention enables, by the choice of suitable reaction conditions, for a low conversion of the alcohols used, which leads to an increased selectivity of the desired product olefins. Almost no or hardly any isomers of the product olefins form. Furthermore, the phase separation allows for a recycle of unreacted alcohols or dialkyl ethers formed into the dehydrogenation reaction. The recycling of the dialkyl ethers formed as intermediates and of the unreacted alcohol, particularly low, more easily achievable conversions can be run in the dehydration without getting economic disadvantages.

The resulting isomers of the product olefins, the quantitative amount of which, as already stated, is usually very low, are either removed or fed into an isomerization unit. Furthermore, it is also conceivable to keep the isomers of the product olefins in the product olefins. Preferably, the isomers of the product olefin are, in the isomerization unit, at least partially converted to the product olefin. Furthermore, the undesired isomers of the product olefins can at least partially be refed into the dehydration unit where they are at least partially converted. In a recycle, however, care but must be taken to ensure that the isomers of the product olefins do not accumulate in the circulation.

Furthermore, by the choice of dehydration conditions which are suitable to provide a low conversion, the further isomerization step common in the prior art can be omitted. With a low conversion, unwanted by-products such as isomers of the desired product olefin(s) and other alkanes or alkenes are produced only in a very reduced level. In a low conversion, besides the desired product olefins, a considerable number of dialkyl ether compounds as intermediates are formed. The latter can be recycled, as described. Thus they are available to a further dehydration step as reactants. Furthermore, from a low conversion the desired product olefin results in a high product purity. The olefin stream can be supplied in many cases to a consumer without any further working up.

Alternatively, the olefin stream can also be fed to an isomer separation unit in which the olefin stream is subject to a further purification and separation. This can for example be done by means of a rectification. In the isomer separation unit, by-products still present can be separated from the olefin stream. Furthermore, in the isomer separation unit, a separation of the desired product olefin from the possibly produced isomers of the product olefin is performed. For example, 1-butene can be separated from 2-butene that may have been formed from the olefin stream. The desired product olefin separated in the isomer separation unit, such as 1-butene, can be supplied to a consumer.

In some methods, the undesired isomer is then converted in an isomerization unit. In the isomerization unit, then a partial conversion of the separated isomer to the desired product olefin is performed. For example, 2-butene is partially isomerized to 1-butene. The isomeric mixture formed in the isomerization unit can then—after a cooling—be fed into the isomer separation unit for the separation of the desired product olefin from the isomer mixture. This allows the yield and selectivity to the desired product olefin to be increased.

For an isomerization, additional energy for the necessary isolation of the desired product olefin from the isomer mixture of the isomerization unit is necessary, because the mixture of isomers must be supplied to the isomer separation unit for the isolation of the desired product olefin. Furthermore, the isomerization requires energy since it is operated at ca. 400° C. Since the separations in the isomer separation unit generally take place in a temperature range from 30° C. to 100° C., an unit and energy additionally would have to be provided which heat the mixture of isomers to about 400° C., starting from this temperature range.

In carrying out the process according to the invention, the proportion to be isomerized is very low or virtually non-existent. If the processing of the isomers is eliminated, the apparatus and energy expenditure described above can be completely eliminated. Even with a processing of the isomers, a significantly lower energy expenditure would be necessary in the method of the present invention, since the isomeric proportion is much lower due to the high selectivity and yield. The energy required to bring the “recycle products”, that is for example unreacted butanol and the dibutyl ether formed, to the dehydration temperature is below the additional energy consumption for the workup of isomers otherwise necessary.

Another advantage of using the method of the invention is that the apparatus expense required in terms of the dehydration unit can be further reduced. In general, the dehydration unit consists of a series connection of at least one fixed-bed reactor, in particular two or three reactors are needed in principle to achieve a complete conversion. However, since in the present process no full conversion is necessary and the reaction conditions are chosen in the dehydration unit such that a low conversion takes place, a reactor in the dehydration unit can be advantageously eliminated.

As catalysts for the dehydration step preferably inorganic ceramic catalysts, especially ZrO₂, zeolites, Al₂O₃ or aluminosilicates are used. However, other suitable catalysts may be employed as well.

In one embodiment of the invention, the isomers that are separated in the isomer separation unit are recycled to the educt stream. The isomers thus separated are preferably fed to the educt stream if the dehydration is performed by means of an Al₂O₃ or aluminosilicate catalyst. When using such a catalyst in the dehydration step, also an isomerization of the isomer of the desired product olefin to the product olefin is performed. In particular, 2-butene separated in the isomer separation unit is preferably recirculated to the dehydration, to be isomerized there by means of a catalyst, in particular an Al₂O₃ catalyst, to 1-butene.

In a further embodiment of the invention the isomers of the desired product olefin can also be recycled to the dehydration, to act as a heat transfer medium or to influence the balance between the desired product olefin and the corresponding isomer of the desired product olefin. In particular, there is a influencing of the balance between 1-butene and 2-butene in favour of 1-butene.

The dehydration is designed such that low conversions of the alcohol are effected. This is achieved by the choice of parameters discussed in the following.

Advantageously, the dehydration is performed at a temperature between 200° C. and 500° C., particularly between 280° C. and 400° C., particularly preferably between 300 and 360° C.

In one embodiment, the dehydration is performed at a space velocity (liquid hourly space velocity, LHSV) of 1 h⁻¹ to 15 h⁻¹, especially from 2 h⁻¹ to 10 h⁻¹, particularly preferably of 3 h⁻¹ to 9 h⁻¹.

Under LHSV (liquid hourly space velocity), in the context of the present invention, the ratio of the feed to the dehydration (measured in m³/h) and the catalyst volume (measured in m³) is to be understood.

In one embodiment, the dehydration is carried out at a pressure in a range of from 3 bar to 30 bar, in particular from 5 bar to 17 bar, particularly preferably from 6 bar to 10 bar.

In a further embodiment of the invention, the phase separation is performed in a range from 3 bar to 30 bar, in particular from 5 bar to 17 bar, particularly preferably from 6 bar to 10 bar.

In one embodiment of the invention, the separation is performed in a range from 3 bar to 30 bar, in particular from 5 bar to 17 bar, particularly preferably from 6 bar to 10 bar.

In one embodiment, the pressure in the dehydration, the phase separation and/or the separation is in a range from 3 bar to 30 bar, in particular from 5 bar to 17 bar, particularly preferably from 6 bar to 10 bar.

In one embodiment, the pressure in the dehydration, the phase separation and the separation is in the range of 5 bar to 17 bar, in particular from 6 to 10 bar, wherein butanoles or higher alcohols are used as educts.

The pressures referred to above in regard to dehydration, phase separation, and the separation can be selected from the respective ranges independently of each other, so that the dehydration, the phase separation and the separation are carried out at different pressures. Alternatively, the pressures selected from the above ranges can also include the same values for the dehydration and/or the phase separation and/or the separation.

In one embodiment of the invention, the phase separation is performed at a temperature between −10° C. and 90° C., especially between 20° C. and 90° C., especially preferably between 30° C. and 50° C.

Preferably, with the method according to the invention, essentially a 1-alkene, especially 1-butene, is produced. Furthermore, other corresponding alkenes can be produced by the method according to the invention.

In the following, the invention shall be described with reference to figures of particularly advantageous embodiments. In the drawings,

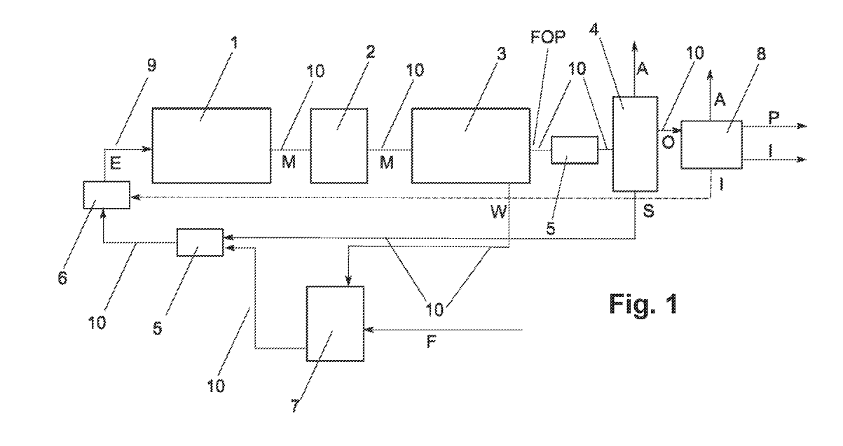

FIG. 1 shows an embodiment of the inventive method for producing 1-butene;

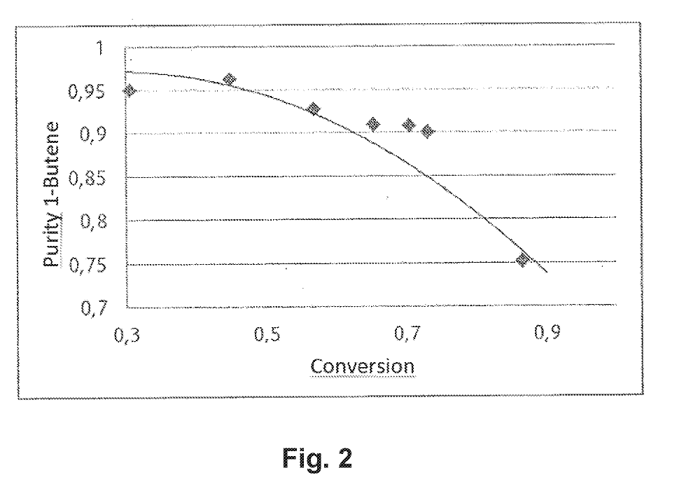

FIG. 2 shows the purity of the formed product olefin 1-butene in relation to a certain conversion rate;

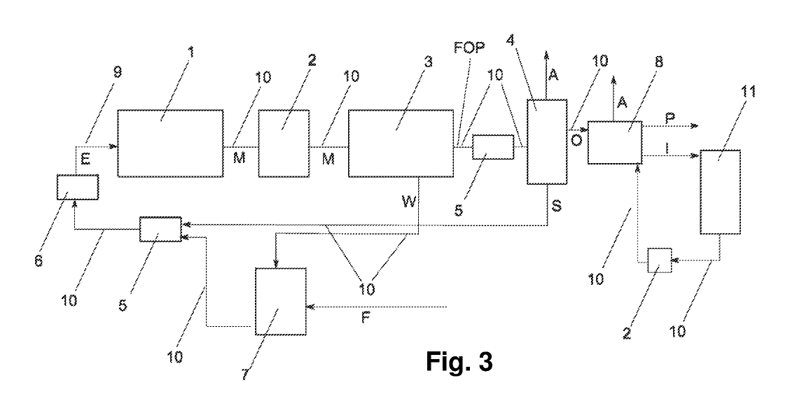

FIG. 3 shows the process of the invention of FIG. 1, taking into account the energy-intensive steps;

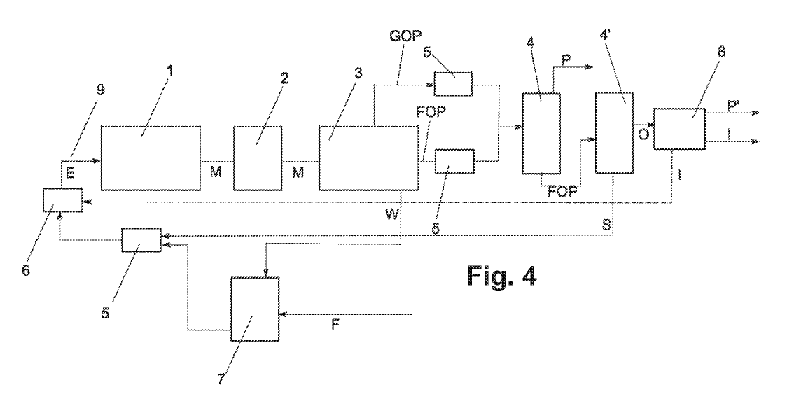

FIG. 4 shows an embodiment of the inventive method when using an alcohol mixture comprising lower and higher alcohols (3-phase separation).

FIG. 1 shows a particularly advantageous embodiment of the method according to the invention. A butanol-water mixture from a fermentation process (not shown here for reasons of clarity) is fed into a enrichment unit 7. Therein, an enrichment of the alcohol content of the butanol-water mixture is performed by means of distillation, excess water being withdrawn from the separation unit 7. The butanol-water mixture correspondingly enriched forms an educt stream E which is passed from the enrichment unit 7 into a compression unit 5.

The educt stream E is compressed in the compression unit 5, such as a pump, to a pressure of 5 bar to 17 bar and is then fed into a heating unit 6 where the educt stream is heated to a temperature of 300° C. to 360° C.

Thereafter, the compressed and superheated educt stream is fed into the dehydration unit 1. The dehydration can include one or more reactors (not shown here for the sake of clarity). In the dehydration unit 1 the dehydration of the butanol occurs. The reaction conditions in the dehydration unit are selected such that only a low conversion rate of the butanol used to the desired product olefin 1-butene is present. Under the given reaction conditions, the corresponding dibutyl ether is preferably generated in addition to the desired 1-butene. The isomer (2-butene) to the product olefin 1-butene is formed only in a very small range. Other by-products, such as for example other C₄ hydrocarbons, can be observed only in traces.

After dehydration, the reaction products are passed into a cooling unit 2. In this context, the pressure of the dehydration has been chosen such that after cooling to about 40° C. essentially a two-phase mixed reaction product stream M is formed. An organic liquid phase FOP is formed which essentially comprises unreacted butanol, dibutyl ether, the desired product olefin 1-butene and its corresponding isomer 2-butene (the latter in small amounts). The second phase formed is an aqueous phase which is present in liquid form and which is substantially comprises—because of limited solubility—small amounts of butanol and a lower amount of the dibutyl ether formed.

The two-phase mixed reaction product stream M that is formed is passed out of the cooling unit 2 and into a phase separation unit 3. In the phase separation unit 3, at a temperature of 40° C. and a pressure of 3 bar to 16 bar, a separation of the two liquid phases is carried out. This can for example be done by means of a decanter. The aqueous phase is fed into the concentration unit 7. Thus, a portion of the unreacted butanol (and small amounts of the dibutyl ether formed) are combined in the enrichment unit 7 with the butanol-water mixture from the fermentation (fermentation stream F), enriched by removal of water therein, and subsequently fed to the educt stream E. Alternatively, the aqueous phase can, after passing through the enrichment unit 7, be combined with an educt stream E wherein the alcohol (or the alcohols) are provided from synthesis gas (not shown here). Thus, no contaminated waste water stream is formed in the dehydration step, but the water obtained there is fed to the enrichment step. Thereby, the water obtained during dehydration, which contains the reactants, is refed into the reaction cycle. Only substantially pure water separated off from the enrichment unit 7 is obtained.

The organic liquid phase FOP separated in the phase separation unit 3 can be conveyed via a pump into a downstream separation unit 4. In an alternative that is not shown here also an organic gaseous phase can be present. This can be passed via a compressor into the separation unit. The other steps are essentially valid analogously.

Advantageously, the pressure of the dehydration step can be chosen such that it is already high enough to carry out the separation steps in the separation unit 4, whereby thus the compression unit can be omitted. In the separation unit 4, the resulting alkenes (1-butene and 2-butene) are separated from the unreacted butanol still contained in the organic liquid phase FOP (with higher alcohols such as e.g. butanol, the major part of the unreacted alcohol is typically contained in the organic liquid phase) and from the dibutyl ether formed as intermediate product and from the other by-products. The unreacted butanol and the dibutyl ether formed form a separation stream S which is passed into the compression unit 5, where it is mixed with the enriched reactant mixture from the enrichment unit 7, compressed and then fed into the educt stream E.

Furthermore, the by-products contained in the organic liquid phase FOP, such as for example carbon monoxide, hydrogen, carbon dioxide, methane or alkanes are additionally separated in the separation unit 4 and withdrawn.

The product olefins P (1-butene and 2-butene) separated from the organic liquid phase FOP in the separation unit 4 can be passed into an isomer separation unit 8. Therein, a further separation of by-products that still may be present (as described earlier) takes place. In particular, in the isomer separation unit 8, a separation of the desired product olefin 1-butene from the corresponding isomer 2-butene takes place. The isolated 1-butene can be supplied to a consumer.

The 2-butene formed in the process according to the invention is so low in its amount that an energy-intensive isomerization of 2-butene to the desired 1-butene is most often not necessary. Alternatively, it is (as shown in FIG. 4) possible that the separated 2-butene is fed into an isomerization unit 11, wherein it is isomerized into a reaction mixture consisting of 1-butene and 2-butene which is then again passed for a further separation into the isomer separation unit 8 (after cooling to the desired isomer separation temperature of 30° C. to 100° C.). There, again a separation of 1-butene and 2-butene takes place, 2-butene in turn being passed into the isomerization unit 11.

Alternatively, if the dehydration takes place by means of a catalyst selected from the group consisting of Al₂O₃ or aluminosilicates, the separated isomers I can be fed into the educt stream E. These catalysts are capable of converting the separated isomers during dehydration into the product olefins (this alternative is represented by a dashed line in the Figure).

In a further alternative, the isomers may be fed, when the alcohols have been produced synthetically, into a reformer (not shown here).

In the process of the invention, no wastewater stream is present which has to be disposed of separately. Rather, the unreacted butanol fraction and the intermediates formed (dibutyl ether) are refed via the phase separation unit 3 and the separation unit 4 to the educt stream E for a further conversion in the dehydration unit 1. Due to this re-use and due to the low conversion in the dehydration unit 1, a high selectivity and yield of 1-butene is achieved. By the specific choice of suitable reaction conditions, it is thus possible by the method according to the invention to enhance both the selectivity and the yield of 1-butene, without the use of an isomerization step being necessary. With the inventive method, it is possible to increase, for example, the yield of 1-butene by ca. 20%, wherein the dehydration is carried out up to a conversion of ca. 50%. The methods known in the prior art basically target a conversion of ca. 90% in the dehydration. Considering the isolated butene mixtures (1-butene and 2-butene), the inventive method allows for a 1-butene content of about 95%. In the prior art (US Patent 2011/0213104 A1), for example, a 1-butene content of 77.5% and a 2-butene content of 20% is obtained.

Alternatively, other alcohols or alcohol mixtures can be used.

FIG. 2 shows the purity of the resulting butene mixture (1-butene and 2-butene) after separation from a separation unit 4 with respect to the conversion being present in the dehydration reaction. As is apparent from FIG. 3, at a conversion of less than 50%, a purity of over 95% of 1-butene can be obtained. Even at a conversion of about 70%, a purity of about 90% can still be obtained with respect to the desired 1-butene obtained.

FIG. 3 shows the inventive method when using an isomerization unit 11, wherein the necessary energy input has been taken into account. For elements that have been provided with the same reference numerals, reference is made to the explanation of FIG. 1. By an isomerization, in isomerization unit 11 an increased energy expenditure is present since the isomerization unit 11 must be operated at ca. 400° C. to achieve a suitable isomerization. The isomerization mixture must then, before it is fed back to the isomer separation unit 8, be cooled to a range from 30° C. to 100° C. Also the additional separation step of isomer mixture provided in the isomer separation unit 8 requires further energy to provide a separation, for example by means of distillation, of the desired product olefin 1-butene from the corresponding isomer 2-butene.

By using the method according to the invention, the proportion to be isomerized is considerably lower. This allows for a high energy saving. The additional energy expenditure which is necessary to cool the unreacted butanol and the resulting dibutyl ether separated via the phase separation or the separation after the dehydration is less than the energy expenditure that would be necessary for a complete isomerization.

Furthermore, the apparatus expense can be reduced by the method according to the invention. Typically, a dehydration is performed in a series connection of the fixed-bed reactors, in particular of at least two or three reactors, in order to achieve a full conversion. Since no full conversion is necessary according to the invention but a low conversion rate is desired, a reactor can usually be omitted in the dehydration unit.

FIG. 4 shows the inventive method when using an alcohol mixture of lower and higher alcohols (3-phase separation). In essence, the process is carried out analogously to the process described in FIG. 1. In the present case, only the differences are discussed. For elements that are provided with the same reference numerals reference is made to the explanation of FIG. 1 and FIG. 3.

Through the use of such alcohol mixture, after the dehydration and cooling, in the phase separation unit 3 an aqueous phase W, an organic liquid phase FOP and an organic gaseous phase GOP are present. In the phase separation unit 3, the three phases are separated from another.

The aqueous phase W is passed into the enrichment unit 7 for concentration and is then fed into the educt stream E. The organic liquid phase FOP is fed by means of a compression unit 5, in this case a pump, and the organic gaseous phase GOP is fed by means of another compression unit 5, in this case a compressor, into a first separation unit 4.

In the first separation unit 4, the product olefins P are separated from the by-products and are withdrawn. If necessary, the product olefins P can be fed into a further separation which separates the product olefins different from each other.

The remaining organic-liquid phase FOP is passed to a second separation unit 4′ in which the product olefins P′ that are formed and that are still contained in the organic liquid phase FOP are separated from the unreacted higher alcohols and optionally from dialkyl ethers (and optionally further by-products that are still present). The unreacted alcohols and the dialkyl ethers formed form a separation stream S, which is passed into the compression unit 5 where it is mixed with the enriched reactant mixture coming from the enrichment unit 7, compressed and then fed into the educt stream E.

The product olefins P′ separated from the organic liquid phase FOP in the separation unit 4′ are passed into an isomer separation unit 8. Therein, a further separation of optionally still present by-products (as described earlier) takes place. In particular, in the isomer separation unit 8, a separation of the desired product olefin P from the corresponding isomers takes place.

For the sake of clarity, the exhaust stream A and the withdrawal systems 10 are not shown. Reference is made to FIGS. 1 and 3.

By choosing an appropriate pressure of the dehydration at least one compressor can be omitted after the phase separation.

### LIST OF REFERENCE NUMERALS

| | |
|---|---|
| 1 | Dehydration unit |
| 2 | Cooling unit |
| 3 | Phase separation unit |
| 4 | Separation unit |
| 5 | Compression unit |
| 6 | Heating unit |
| 7 | Enrichment unit |
| 8 | Isomer separation unit |
| 9 | Introduction unit |
| 10 | Withdrawal system |
| 11 | Isomerization unit |
| A | Exhaust stream |
| E | Educt stream |
| I | Isomer of the product olefin |
| F | Fermentation stream |
| M | Mixed reaction product stream |
| O | Olefin stream |
| FOP | Organic liquid phase |
| GOP | Organic gaseous phase |
| P | Product olefin |
| S | Separation stream |
| W | Aqueous phase |

1. A method for producing product olefins by catalytic dehydration, comprising the steps of:
feeding an educt stream (E) comprising an alcohol-water mixture into a dehydration unit (1), wherein the alcohol-water mixture comprises at least one alcohol and water,
converting the reactants contained in the educt stream (E) in the dehydration unit (1) by catalytic dehydration to form a mixed reaction product stream (M),
characterized in that
the dehydration conditions in the dehydration unit (1) are selected such that the unreacted at least one alcohol in the mixed reaction product stream (M) comprises an alcohol content ranging from 20 wt %-80 wt %.

2. A method according to claim 1, characterized in that the mixed reaction product stream (M) is cooled, so that a mixed reaction product stream (M) with several phases is formed, comprising an aqueous phase (W) and a organic liquid phase, wherein a phase separation (3) of the aqueous phase (W) from the organic-liquid phase is carried out.

3. A method according to claim 2, characterized in that at least partial streams of the aqueous and/or of the organic liquid phase are recycled to the feed stream.

4. A method according to claim 2 or 3, characterized in that an organic gaseous phase is additionally formed.

5. A method according to one of claims 2 to 4, characterized in that subsequently to the phase separation (3) of the aqueous from the organic-liquid phase,
a. the aqueous phase (W) is fed to the educt stream (E) via a concentration (7), and/or
b. the organic liquid phase is passed into at least one separation unit (4), wherein unreacted alcohol and dialkyl ethers formed in the dehydration are separated such that they form a separation stream (S) which is fed to the educt stream (E).

6. A method according to one of claims 1 to 5, characterized in that in the separation unit (4) the product olefins (P), and optionally the isomers (I) thereof, are separated from the by-products and the compounds which form the separation stream (S), wherein the product olefins (P), and optionally the isomers (I) thereof, form then an olefin stream (O) which is withdrawn from the separation unit (4).

7. A method according to claim 4, characterized in that the olefin stream (O) is fed into a isomer separation unit (8) for separating the isomers (I) of the product olefins from the product olefins (P), and that the separated isomers (I) are fed into
an isomerization unit (11), or
the dehydration unit (1), or
a reformer.

8. A method according to one of claims 1 to 7, characterized in that the dehydration is performed at a temperature between 200° C. and 500° C.

9. A method according to one of claims 1 to 8, characterized in that the dehydration is performed at a space velocity (LHSV) of 1 h⁻¹ to 15 h⁻¹.

10. A method according to one of the preceding claims, characterized in that the at least one alcohol is selected from the group of C₃-C₈ alcohols.

11. A method according to one of the preceding claims, characterized in that the educt stream (E) comprises an alcohol content of 5 wt %-98 wt %.

12. A method according to one of the preceding claims, characterized in that the pressure in the dehydration is in a range from 3 bar to 30 bar.
